(19) United States
(12) Patent Application Publication
(10) Pub. No.: US 2025/0114559 A1
(43) Pub. Date: Apr. 10, 2025

(54) CONTROL DEVICE AND SLEEP INDUCTION DEVICE

(71) Applicants: KYOCERA Corporation, Kyoto-shi, Kyoto (JP); University of Tsukuba, Tsukuba-shi, Ibaraki (JP)

(72) Inventors: Junki ENDO, Kyoto-shi, Kyoto (JP); Takashi ABE, Tsukuba-shi, Ibaraki (JP); Yunyao ZHU, Tsukuba-shi, Ibaraki (JP)

(21) Appl. No.: 18/730,192

(22) PCT Filed: Jan. 10, 2023
(86) PCT No.: PCT/JP2023/000274

§ 371 Date: Jul. 18, 2024

(30) Foreign Application Priority Data
Jan. 19, 2022 (JP) 2022-006550

(51) Int. Cl. A61M 21/02; A61M 21/00

(52) U.S. Cl. CPC A61M 21/02; A61M 2021/0011; A61M 2021/0033; A61M 2205/50; A61M 2230/005

(57) ABSTRACT

To improve the quality of sleep. A control device includes a determiner that determines a stage of sleep of a subject on the basis of biological information on the subject, and a controller that controls a sleep induction device including a beat generator that generates a binaural beat. The controller causes the beat generator to generate a binaural beat having a frequency lower than 1.0 Hz at least in the stage of sleep of the subject.

### TECHNICAL FIELD

The present disclosure relates to a sleep induction device and a control device that controls the sleep induction device.

### BACKGROUND OF INVENTION

Patent Document 1 discloses a technique for generating a series of binaural beats included in a sleep program from a pair of speaker units. Frequencies of the series of binaural beats may include frequencies corresponding to, for example, the α, δ and θ frequency bands.

### CITATION LIST

### Patent Literature

Patent Document 1: JP 2014-519937 T

### SUMMARY

According to an aspect of the present disclosure, a control device includes: a determiner configured to determine a stage of sleep of a subject on the basis of biological information on the subject; and a controller configured to control a sleep induction device including a beat generator configured to generate a binaural beat, in which the controller is configured to cause the beat generator to generate a binaural beat having a frequency lower than 1.0 Hz at least in the stage of sleep of the subject.

A sleep induction device according to an aspect of the present disclosure includes a beat generator configured to generate a binaural beat having a frequency lower than 1.0 Hz at least in a stage of sleep.

### BRIEF DESCRIPTION OF THE DRAWINGS

FIG. 1 is a block diagram illustrating an example of a schematic configuration of a sleep induction device.

FIG. 2 is a flowchart showing an example of a flow of processing by a control device.

FIG. 3 shows graphs of verification results.

### DESCRIPTION OF EMBODIMENTS

Hereinafter, control of binaural beats generated in the brain of a subject, according to the present disclosure, will be described. Note that when “A to B” is described in the present specification, it indicates “A or more and B or less”.

### [Binaural Beats]

Binaural beats refer to a sound having a frequency corresponding to a difference between two frequencies and generated in the brain of a subject by causing the subject to listen to sounds having the two different frequencies from the left and right ears. It is known that the brain waves are synchronized with the frequency corresponding to the difference.

Based on this finding, the present inventors have made intensive studies, focusing on frequencies of binaural beats for causing a subject to fall asleep efficiently. As a result, the present inventors have found that sleep-onset latency can be shortened by binaural beats having frequencies lower than 1.0 Hz. The present inventors have found that a time to reach a deep sleep stage can be shortened by binaural beats having frequencies lower than 1.0 Hz. Hereinafter, this time is referred to as deep sleep latency. In particular, the present inventors have found that binaural beats having frequencies of 0.2 Hz to 0.3 Hz can more effectively shorten the sleep-onset latency or the deep sleep latency. Among them, the present inventors have found that binaural beats having a frequency of 0.25 Hz can more effectively shorten the sleep-onset latency or the deep sleep latency.

Herein, stages of sleep can be generally classified into three stages, namely, wakefulness, REM sleep, and non-REM sleep. Non-REM sleep can be further classified into stage 1 (N1), stage 2 (N2), and stage 3 (N3) from the lightest stage of sleep. That is, the stages of sleep including non-REM sleep can be classified into stages 1, 2, and 3 in order from the lightest stage of sleep. REM sleep is sleep involving rapid eye movement (REM). Non-REM sleep is sleep involving no rapid eye movement. The deep sleep stage corresponds to stage 3.

The above classification may be performed on the basis of brain wave data detected by an electroencephalograph worn on the subject. Brain waves are classified into four types of β waves, α waves, θ waves, and δ waves in ascending order of wavelength. The β wave is a brain wave with a frequency of about 38 to 14 Hz, for example. The α wave is a brain wave with a frequency of about 14 to 8 Hz, for example. The θ wave is a brain wave with a frequency of about 8 to 4 Hz, for example. The δ wave is a brain wave with a frequency of about 4 to 0.5 Hz, for example.

A person is asleep when the θ wave and the δ wave are dominant relative to the β wave and the α wave. Here, the expression “are dominant” means that the percentage of a certain wave is large in the measured brain waves. The dominant brain wave is known to periodically change in the range of the θ wave and the δ wave during sleep. When the percentage of the θ wave included in the brain waves is less than a predetermined value, a person is in the state of REM sleep, and when the percentage of the θ wave is equal to or greater than a predetermined value and when the δ wave is dominant, a person is in the state of non-REM sleep. Stage 1 represents, for example, a state in which the α wave is equal to or less than 50%, and various low-amplitude frequencies are mixed. Stage 2 represents, for example, a state in which an irregular low-amplitude θ wave and δ wave appear, but no high-amplitude slow wave exists. Stage 3 represents, for example, a state in which a slow wave of equal to or less than 2 Hz and 75 μV is 20% or more. A state in which a slow wave of equal to or less than 2 Hz and 75 μV is 50% or more may be referred to as stage 4.

The present inventors have made intensive studies, and as a result have found the following. That is, the present inventors have found that the sleep efficiency may deteriorate, for example, wake after sleep onset may be caused, when binaural beats having frequencies lower than 1.0 Hz are continuously generated without changing the frequencies in order to shorten the sleep-onset latency or the deep sleep latency. The present inventors have found that the sleep efficiency may also deteriorate when a sound for generating binaural beats is continuously generated without reducing a volume thereof. The present inventors have found that the sleep efficiency may also deteriorate even in the stage of sleep of stage 3 in particular, if binaural beats having frequencies lower than 1.0 Hz are continuously generated, or if sounds are continuously generated without reducing the volume.

The present inventors have also found that the sleep efficiency tends to be improved in a state in which frequencies are set to 0 Hz, particularly in a state in which sounds for generating binaural beats are stopped, as compared with a state in which binaural beats are being generated.

The above-described findings (technical idea) are newly obtained by the present inventors, and are neither disclosed nor suggested in, for example, Patent Document 1. That is, for example, Patent Document 1 does not disclose or suggest the setting of the frequencies of the binaural beats studied from the standpoint of shortening the sleep-onset latency or the deep sleep latency. For example, Patent Document 1 does not disclose or suggest the settings of the frequencies of the binaural beats and the sound for generating the binaural beats, which have been studied from the standpoint of reducing the deterioration in sleep efficiency while shortening the sleep-onset latency or the deep sleep latency. An aspect of the present disclosure is to control binaural beats to be generated in consideration of the above-described findings. Hereinafter, one embodiment will be described.

### Sleep Induction Device

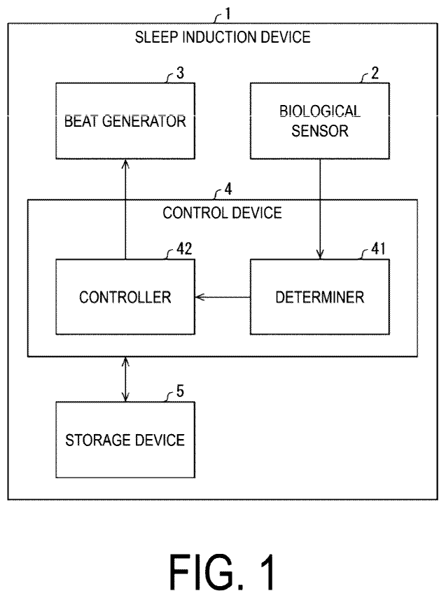

FIG. 1 is a block diagram illustrating an example of a schematic configuration of a sleep induction device 1. The sleep induction device 1 is a device that induces a subject to a stage of sleep by binaural beats. As illustrated in FIG. 1, the sleep induction device 1 may include a biological sensor 2, a beat generator 3, a control device 4, and a storage device 5. However, the sleep induction device 1 may include at least the beat generator 3. In this case, for example, the sleep induction device 1 may be communicably connected to the biological sensor 2 and/or the control device 4 provided outside the sleep induction device 1.

### Biological Sensor

The biological sensor 2 is a sensor that detects biological information on the subject. Examples of the biological information to be detected by the biological sensor 2 may include blood flow information, electrocardiogramnformation, respiratory information, perspiration information, body temperature information, body motion information, and brain wave information. The biological sensor 2 transmits the biological information to the control device 4 in order to determine a stage of sleep of the subject.

In a case where the biological sensor 2 detects blood flow information, the biological sensor 2 may be a blood flow meter such as a laser Doppler blood flow meter, an ultrasonic blood flow meter, or a pulse wave meter. Examples of the pulse wave meter may include a photoplethysmography meter. The blood flow information may include, for example, at least one selected from the group consisting of an amount of blood flow, a heart rate, a heartbeat interval, a cardiac output, a blood flow wave height, and a coefficient of variation of blood vessel motion (vasomotion).

The amount of blood flow represents an amount of blood flowing through a blood vessel of a unit volume per unit time. The heart rate represents the number of beats of the heart per unit time. The heartbeat interval represents an interval between beats of the heart. Cardiac output represents the amount of blood delivered in one beat of the heart. The blood flow wave height represents a difference between the maximum value and the minimum value of the amount of blood flow in one beat of the heart. Vasomotion represents a contraction-expansion movement of the blood vessel that occurs spontaneously and rhythmically. The coefficient of variation of vasomotion represents a value indicating, as a variation, the change in the amount of blood flow occurring on the basis of the vasomotion.

In a case where the biological sensor 2 detects electrocardiogramnformation, the biological sensor 2 may be an electrocardiogram a case where the biological sensor 2 detects respiratory information, the biological sensor 2 may be a sensor such as a microphone and an accelerometer. When a microphone is used as the biological sensor 2, the biological sensor 2 can detect information indicating a respiratory sound as the respiratory information. When an acceleration sensor is used as the biological sensor 2, the biological sensor 2 can detect information indicating motion of the chest as the respiratory information.

In a case where the biological sensor 2 detects perspiration information, the biological sensor 2 may be a perspiration meter that detects an amount or weight of perspiration as the perspiration information. In a case where the biological sensor 2 detects body temperature information, the biological sensor 2 may be a thermometer such as a thermistor, an infrared sensor, or a mercury thermometer. In a case where the biological sensor 2 detects body motion information, the biological sensor 2 may be an accelerometer or a pressure gauge. In a case where the biological sensor 2 detects brain wave information, the biological sensor 2 may be an electroencephalograph.

### Control Device

The control device 4 may determine a stage of sleep of the subject using at least one piece of biological information among the biological information described above. Therefore, the sleep induction device 1 needs to include at least one biological sensor 2 that can detect biological information used by the control device 4 to determine a stage of sleep.

The beat generator 3 is a device that generates binaural beats. The beat generator 3 may be a pair of headphones or a pair of earphones that convert an audio signal received from the control device 4 into a sound and output the sound. The beat generator 3 need not be a device to be worn on the subject, and may be provided to bedding such as a bed or a pillow. In this case, the beat generator 3 may be disposed in the vicinity of the left and right ears when the subject lies on the bed.

The control device 4 may be a device that integrally controls each unit of the sleep induction device 1. For example, the control device 4 may determine the stage of sleep of the subject on the basis of the biological information on the subject detected by the biological sensor 2. In accordance with the determined stage of sleep of the subject, the control device 4 may cause the beat generator 3 to generate binaural beats or stop the binaural beats generated by the beat generator 3. The control device 4 may include a determiner 41 and a controller 42.

### Determiner

The determiner 41 determines the stage of sleep of the subject on the basis of the biological information on the subject. The determiner 41 transmits a result of the determination to the controller 42.

The determiner 41 may determine the stage of sleep of the subject on the basis of, for example, brain wave information as the biological information. As described above, the stage of sleep can be determined on the basis of brain waves. The determiner 41 may determine the stage of sleep of the subject by using a learned model.

The learned model is obtained by training a mathematical model that imitates neurons of the human cranial nervous system, such that the stage of sleep of the subject can be determined. The mathematical model is a neural network including an input layer, a hidden layer, and an output layer. The mathematical model may be, for example, a convolutional neural network (CNN), a recurrent neural network (RNN), or a long short term memory (LSTM).

The learned model may be constructed by, for example, inputting each of a plurality of types of teacher data to the mathematical model to update weights so as to minimize an objective function. The teacher data may be data in which each of a plurality of types of known biological information is associated with a stage of sleep of the subject at the time when the biological information is detected.

The determiner 41 can determine a stage of sleep of the subject by inputting the biological information received from the biological sensor 2 to the learned model constructed as described above.

The determiner 41 may process and use the received biological information. For example, the determiner 41 may determine the stage of sleep of the subject on the basis of a frequency spectrum obtained by executing frequency analysis processing on the biological information. Examples of the biological information may include blood flow information. Examples of the frequency analysis processing may include Fourier transform processing and wavelet transform processing. The determiner 41 may also use the learned model in the determination. Also in this case, for example, teacher data may be used to construct the learned model. The teacher data may be data in which a stage of sleep of the subject at the time when the biological information is detected is associated with the frequency spectrum obtained by executing the frequency analysis processing on each of the plurality of types of known biological information.

The above-described learned model may be generated by a model generation device that generates a learned model, or may be generated by the control device 4.

### Controller

The controller 42 may control the beat generator 3. Specifically, the controller 42 may cause the beat generator 3 to generate binaural beats having frequencies lower than 1.0 Hz at least in the stage of sleep of the subject.

The controller 42 may transmit an audio signal for causing binaural beats having frequencies lower than 1.0 Hz to be generated to the beat generator 3, for example, in an awake state or when the state is determined to have transitioned from the awake state to a REM sleep state.

When transmitting the audio signal to the beat generator 3, the controller 42 may select one piece of sound source data from a plurality of types of sound source data stored in the storage device 5 and acquire the selected sound source data from the storage device 5. In this case, at least one piece of sound source data may be stored in the storage device 5. The controller 42 may acquire sound source data from an external sound source instead of acquiring sound source data from the storage device 5. In this case, sound source data need not be stored in the storage device 5.

The controller 42 may generate transition data in which a frequency of the acquired sound source data is shifted within a range less than 1.0 Hz. The controller 42 may transmit the acquired sound source data and the transition data in which the frequency of the acquired sound source data is shifted within a range less than 1.0 Hz to the beat generator 3 as the audio signal. Specifically, the controller 42 may transmit the acquired sound source data to the pair of headphones and/or the pair of earphones, as a first audio signal. The controller 42 may transmit the transition data in which the frequency of the acquired sound source data is shifted within a range less than 1.0 Hz to the other of the pair of headphones or earphones as a second audio signal. Thus, the beat generator 3 may generate binaural beats having frequencies lower than 1.0 Hz.

The sound source data may be a pure sound (sound of a single frequency). In this case, the controller 42 may generate the second audio signal (transition data) by varying the frequency of the pure sound within a range less than 1.0 Hz. The sound source data may be any music. In this case, the controller 42 may generate the second audio signal by modulating the frequency of any music such that it is varied within a range less than 1.0 Hz. The controller 42 may generate two pieces of transition data in which the frequencies of the acquired sound source data are shifted so as to be different from each other within a range less than 1.0 Hz, and set the two pieces of transition data as the first audio signal and the second audio signal, respectively.

When the sound source data is stereo data, the controller 42 may extract monaural data from the stereo data. The controller 42 may transmit, to the beat generator 3, the extracted monaural data as the first audio signal and monaural data (transition data) in which a frequency of the monaural data is shifted within a range less than 1.0 Hz as the second audio signal. The controller 42 may generate two pieces of transition data in which frequencies of the extracted monaural data are shifted so as to be different from each other within a range less than 1.0 Hz, and transmit the two pieces of transition data to the beat generator 3 as the first audio signal and the second audio signal, respectively.

When the sound source data is analog data, the controller 42 may convert the analog data into digital data. The controller 42 may transmit, to the beat generator 3, the converted digital data as the first audio signal and digital data (transition data) in which a frequency of the digital data is shifted within a range less than 1.0 Hz as the second audio signal. The controller 42 may generate two pieces of transition data in which the frequencies of the converted digital data are shifted so as to be different from each other within a range less than 1.0 Hz, and transmit the two pieces of transition data to the beat generator 3 as the first audio signal and the second audio signal, respectively.

The controller 42 need not generate the transition data. In this case, the transition data may be stored in advance in the storage device 5 or the external sound source in association with the sound source data. In this case, the controller 42 may transmit the sound source data and the transition data or two pieces of transition data generated in advance from the sound source data to the beat generator 3 as the audio signal.

As described above, it has been found that the sleep-onset latency or the deep sleep latency can be reduced by binaural beats having frequencies lower than 1.0 Hz. Therefore, by causing the beat generator 3 to generate binaural beats having frequencies lower than 1.0 Hz, the sleep-onset latency or the deep sleep latency can be shortened. This can improve the quality of sleep.

The controller 42 may cause the beat generator 3 to generate binaural beats having frequencies of 0.2 to 0.3 Hz. That is, the controller 42 may transmit an audio signal for causing binaural beats having frequencies of 0.2 to 0.3 Hz to be generated to the beat generator 3. Thus, the beat generator 3 may generate binaural beats having frequencies of 0.2 to 0.3 Hz.

As described above, it has been found that the sleep-onset latency or the deep sleep latency can be effectively shortened by binaural beats having frequencies of 0.2 to 0.3 Hz. Therefore, by causing the beat generator 3 to generate binaural beats having frequencies of 0.2 to 0.3 Hz, the sleep-onset latency or the deep sleep latency can be effectively shortened.

### Generation Timing of Binaural Beats

The controller 42 may cause the beat generator 3 to generate binaural beats having frequencies lower than 1.0 Hz at least in a sleep-onset stage.

For example, as described above, the controller 42 may cause the binaural beats to be generated in the awake state. That is, the controller 42 may cause the beat generator 3 to generate the binaural beats before the subject falls asleep. In addition, for example, as described above, the controller 42 may cause the binaural beats to be generated when the state transitions from the awake state to the REM sleep state. That is, when the determiner 41 determines that the stage of sleep of the subject is the sleep-onset stage, the controller 42 may cause the beat generator 3 to generate the binaural beats. In addition, for example, the controller 42 may cause the binaural beats to be generated in the REM sleep state. The determiner 41 may determine the stage of sleep of the subject as the sleep-onset stage in a state in which the state transitions from the awake state to the REM sleep state. The state refers to a state before or after the subject falls asleep. In the following description, it is assumed that the sleep-onset stage refers to any stage between before sleep-onset and after sleep-onset.

In this way, the controller 42 may cause the beat generator 3 to generate binaural beats having frequencies lower than 1.0 Hz before and after the subject falls asleep. This can shorten the sleep-onset latency or the deep sleep latency. In particular, by generating the binaural beats before the subject falls asleep, the sleep-onset latency or the deep sleep latency can be effectively reduced.

The controller 42 may cause the beat generator 3 to generate binaural beats having frequencies lower than 1.0 Hz at a predetermined time. The predetermined time may be set to, for example, a time at which the subject desires to fall asleep. The time may be, for example, midnight. For example, when a predetermined time set in advance is determined to be reached in a state in which the subject is determined to be in the awake state, the controller 42 may cause the beat generator 3 to generate binaural beats having frequencies lower than 1.0 Hz. Accordingly, the controller 42 can cause the binaural beats for shortening the sleep-onset latency or the deep sleep latency to be generated at an effective timing; for example, the controller 42 can cause the binaural beats to be generated when the subject is about to fall asleep at a predetermined time.

The controller 42 may cause the beat generator 3 to generate binaural beats having frequencies lower than 1.0 Hz in accordance with the biological information received from the biological sensor 2. The controller 42 can determine an activity state of the subject on the basis of the received biological information. Therefore, the controller 42 can cause the binaural beats to be generated at an effective timing on the basis of the activity state of the subject in the sleep-onset stage. The sleep-onset stage refers to a state before or after the subject falls asleep.

For example, when α waves are predominantly included in the brain waves detected by the electroencephalograph, the determiner 41 can determine that the subject is in the awake state and that the subject is in a resting state. In addition, for example, when the amount of blood flow detected by the blood flow meter is a predetermined amount, the determiner 41 can determine that the subject is in a resting state. The predetermined amount is, for example, 70 to 80 mm/100 g/min. Therefore, the controller 42 can cause the binaural beats for shortening the sleep-onset latency or the deep sleep latency to be generated at an effective timing; for example, the controller 42 can cause the binaural beats having frequencies lower than 1.0 Hz to be generated when the subject is in a resting state before falling asleep.

### Change in Frequencies of Binaural Beats or Volume of Sound

The controller 42 may change the frequencies of the binaural beats generated in the sleep-onset stage in accordance with the stage of sleep of the subject. As described above, it has been found that when the binaural beats generated in the sleep-onset stage are continuously generated without changing the frequencies thereof, the sleep efficiency may deteriorate. Therefore, when the controller 42 changes the frequencies of the binaural beats in accordance with the stage of sleep of the subject, the likelihood of deterioration of the sleep efficiency can be reduced.

The timing at which the frequencies of the binaural beats are changed and the amount of change in the frequencies are preferably set on the basis of, for example, an experiment such that the sleep-onset latency or the deep sleep latency can be shortened and the likelihood of deterioration of the sleep efficiency can be reduced.

For example, the controller 42 may change the frequencies set at the time of binaural beat generation, only once. For example, when the stage of sleep of the subject transitions to another stage of sleep, the controller 42 may change the frequencies set at the time of binaural beat generation. The controller 42 may change the frequencies set at the time of binaural beat generation, for example, when the REM sleep transitions to the non-REM sleep. The controller 42 may change the frequencies set at the time of binaural beat generation, in a stepwise manner over a plurality of times. The controller 42 may change the frequencies set at the time of binaural beat generation, for example, each time the stage of sleep of the subject transitions to another stage of sleep. The controller 42 may gradually change the frequencies set at the time of binaural beat generation.

For example, the controller 42 may generate new transition data by changing the frequency of the transition data generated most recently by a preset change amount, and transmit the generated transition data to the beat generator 3, as the second audio signal. Thus, the controller 42 may change the frequencies of the binaural beats. The controller 42 may change the frequencies of the binaural beats by changing at least one of the frequencies of the two pieces of transition data in which the frequencies of the sound source data are shifted so as to be different from each other within a range less than 1.0 Hz. The controller 42 may decrease or increase the frequencies of the binaural beats from the frequencies of the binaural beats generated most recently.

The transition data in which the frequencies of transition data in which the frequencies of the sound source data are shifted within a range less than 1.0 Hz may be stored in the storage device 5 or the external sound source in association with the sound source data. The transition data in which at least one of the frequencies of the two pieces of transition data in which the frequencies of the sound source data are shifted so as to be different from each other within a range less than 1.0 Hz is changed may be stored in the storage device 5 or the external sound source in association with the sound source data. A plurality of pieces of transition data in which the frequencies are changed may be stored in the storage device 5 or the external sound source. In this case, the controller 42 may change the frequencies of the binaural beats by acquiring the sound source data and the transition data or the two pieces of transition data from the storage device 5 or the external sound source and transmitting the acquired data as the first audio signal and the second audio signal.

When changing the binaural beats in accordance with the stage of sleep of the subject, the controller 42 may bring the frequencies of the binaural beats close to a specific frequency. A value of the specific frequency and a timing at which the frequency is brought close to the specific frequency may be set on the basis of, for example, an experiment such that the sleep-onset latency or the deep sleep latency can be shortened and the likelihood of deterioration of the sleep efficiency can be reduced. The specific frequency may be, for example, 0.2 to 0.3 Hz. The specific frequency may be, for example, 0.25 Hz. The specific frequency may be a value set in association with each stage of sleep. By bringing the frequency close to the specific frequency set in this way, the controller 42 can effectively reduce the likelihood of deterioration of the sleep efficiency.

For example, when the determiner 41 determines that the stage of sleep of the subject has transitioned from stage 1 or 2 to stage 3, the controller 42 may change the frequencies of the binaural beats generated in the sleep-onset stage. As described above, it has been found that if the binaural beats generated in the sleep-onset stage are continuously generated without changing the frequencies of the binaural beats even in the stage of sleep of stage 3 in particular, the sleep efficiency may deteriorate. For this reason, the controller 42 can more effectively reduce the likelihood of deterioration of the sleep efficiency by changing the frequencies of the binaural beats when the stage of sleep transitions to the stage 3.

The controller 42 may reduce the volume of the sound generated by the beat generator 3 in accordance with the stage of sleep of the subject. Specifically, the controller 42 may reduce the volume of the sound indicated by each of the first audio signal and the second audio signal in accordance with the stage of sleep of the subject. As described above, it has been found that the sleep efficiency may deteriorate if the sound is continuously generated without reducing the volume thereof. For this reason, by reducing the volume in accordance with the stage of sleep, the likelihood of deterioration of the sleep efficiency can be reduced. The timing at which the volume is reduced and the amount of reduction in the volume are preferably set on the basis of, for example, an experiment such that the sleep-onset latency or the deep sleep latency can be shortened and the likelihood of deterioration of the sleep efficiency can be reduced.

For example, when the determiner 41 determines that the stage of sleep of the subject has transitioned from stage 1 or 2 to stage 3, the controller 42 may reduce the volume of the sound generated in the sleep-onset stage. As described above, it has been found that if the sound generated for generating the binaural beats in the sleep-onset stage is continuously generated without changing the volume even in the stage of sleep of stage 3 in particular, the sleep efficiency may deteriorate. For this reason, the controller 42 can more effectively reduce the likelihood of deterioration of the sleep efficiency by changing the frequencies of the binaural beats when the stage of sleep transitions to the stage 3.

The controller 42 may change the frequencies of the binaural beats generated in the sleep-onset stage to 0 Hz in accordance with the stage of sleep of the subject. That is, the controller 42 may stop the binaural beats in accordance with the stage of sleep of the subject. As described above, it has been found that when the frequencies are set to 0 Hz, the sleep efficiency tends to be improved as compared with a case where the frequencies are not changed. Therefore, the controller 42 can more effectively reduce the likelihood of deterioration of the sleep efficiency by stopping the binaural beats.

For example, the controller 42 may set the frequencies of the binaural beats to 0 Hz by setting the frequency of the sound indicated by the first audio signal and the frequency of the sound indicated by the second audio signal to the same value. The frequencies of the sound indicated by the first audio signal and the sound indicated by the second audio signal may have the same value, and may be set to, for example, a value larger than 0 Hz. In this case, the binaural beats are stopped, but the first audio signal and the second audio signal are still provided to the subject. That is, the sound is provided to the subject.

It should be noted that the same value is intended to set the two frequencies to such an extent that significant binaural beats are not generated in the brain of the subject, and is not required to be exactly the same.

On the other hand, the controller 42 may set the frequencies of the binaural beats to 0 Hz by stopping the first audio signal and the second audio signal. That is, the controller 42 may stop the output of the sound generated by the beat generator 3 in the sleep-onset stage. As described above, it has been found that the sleep efficiency tends to be improved when the output of the sound is stopped as compared with a case where the sound is continuously output. Therefore, by stopping the output of the sound, the controller 42 can more effectively reduce the likelihood of deterioration of the sleep efficiency as compared with a case where the binaural beats are stopped while the sound is output.

For example, when the determiner 41 determines that the stage of sleep of the subject has transitioned from stage 1 or 2 to stage 3, the controller 42 may stop the output of the sound generated by the beat generator 3 in the sleep-onset stage. The controller 42 can more effectively reduce the likelihood of deterioration of the sleep efficiency by stopping the output of the sound in the stage of sleep of stage 3.

The storage device 5 can store programs and data that are used by the control device 4. When the determiner 41 does not use the learned model to determine the stage of sleep of the subject, the storage device 5 may store, for example, a table indicating a relationship between the brain waves and the stage of sleep. When the determiner 41 uses the learned model to determine the stage of sleep of the subject, the storage device 5 may store the learned model described above. When the control device 4 functions as a model generation device, the storage device 5 may store a mathematical model, a plurality of types of teacher data, and the like. The storage device 5 may store at least one piece of sound source data. The storage device 5 may store at least one piece of transition data in association with the sound source data.

### Flow of Processing by Control Device

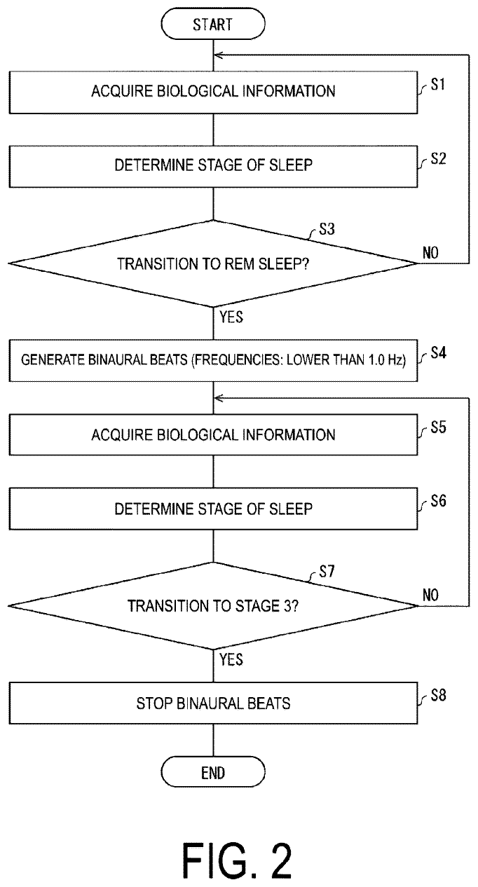

FIG. 2 is a flowchart showing an example of a flow of processing by the control device 4. The biological sensor 2 transmits biological information detected from the subject to the control device 4. Thus, the control device 4 receives the biological information from the biological sensor 2 (S1). The determiner 41 may determine a stage of sleep of the subject on the basis of the biological information received from the biological sensor 2 (S2).

For example, the determiner 41 may determine whether the subject has transitioned from an awake state to a REM sleep state (S3). When the determiner 41 determines that the subject has transitioned to the REM sleep state (YES in S3), the controller 42 may cause the beat generator 3 to generate binaural beats having frequencies lower than 1.0 (S4). Thus, the controller 42 can cause the beat generator 3 to generate the binaural beats in the sleep-onset stage.

When the determiner 41 determines in the processing of S3 that the subject has not transitioned to the REM sleep state (NO in S3), the processing returns to the processing of S1. That is, the control device 4 may execute the processing of S1 and S2 until the determiner 41 determines that the subject has transitioned to the REM sleep state.

However, the controller 42 may cause the beat generator 3 to generate binaural beats having frequencies lower than 1.0 at least in the sleep-onset stage. The controller 42 may cause the beat generator 3 to generate the binaural beats, for example, when a predetermined time set in advance is reached or when the subject is determined to be in a resting state before the sleep-onset on the basis of the biological information.

In addition, as described above, when causing the beat generator 3 to generate the binaural beats before the subject falls asleep, the controller 42 may start from the processing of S4 without executing the processing of S1 to S3. That is, the controller 42 may first cause the binaural beats to be generated in the awake state, and then execute processing of acquiring biological information (corresponding to S5 described below) and processing of determining a stage of sleep (corresponding to S6 described below). This can effectively shorten the sleep-onset latency. In particular, an arrival time from the awake state to the REM sleep state can be effectively shortened.

After the processing of S4, the control device 4 receives the biological information from the biological sensor 2 (S5). The determiner 41 may determine a stage of sleep of the subject on the basis of the biological information received from the biological sensor 2 (S6).

The determiner 41 may determine whether the stage of sleep of the subject has transitioned from stage 1 or 2 to stage 3 (S7). When the determiner 41 determines that the stage of sleep has transitioned to stage 3 (YES in S7), the controller 42 may stop the binaural beats generated by the beat generator 3 (S8).

For example, the controller 42 may stop transmission of the first audio signal and the second audio signal to the beat generator 3. In this case, the controller 42 stops the binaural beats by causing the beat generator 3 to stop outputting the sound. The controller 42 may stop the binaural beats by setting the frequency of the sound of the first audio signal and the frequency of the sound of the second audio signal to the same value. This allows reduction in the likelihood of deterioration of the sleep efficiency due to the continuous generation of the binaural beats without changing the frequencies or the volume during the sleep of the subject.

In S7, the determiner 41 determines whether the stage of sleep transitions from stage 1 or 2 to stage 3, but may determine, for example, transition from the REM sleep to stage 1 or transition from stage 1 to stage 2. That is, the controller 42 may stop the binaural beats in accordance with the stage of sleep of the subject.

The controller 42 need not stop the binaural beats in order to reduce the likelihood of deterioration of the sleep efficiency. The controller 42 may change the frequencies of the binaural beats or reduce the volume of the sound in accordance with the stage of sleep of the subject. The controller 42 may gradually change the frequencies of the binaural beats or gradually decrease the volume of the sound. In particular, in the latter case, after the controller 42 causes binaural beats having frequencies lower than 1.0 Hz to be generated, the processing of S5 and S6 need not be executed.

The control device 4 of the present embodiment enables the sleep-onset latency or the deep sleep latency to be shortened. This can contribute to achievement of Goal 3 “Good health and well-being” of Sustainable Development Goals (SDGs).

### Examples

It was verified whether the sleep-onset latency (N2 latency) and the deep sleep latency (N3 latency) can be shortened by causing a subject to listen to binaural beats having frequencies of 0.2 Hz or higher and 0.3 Hz or lower, particularly 0.25 Hz in the sleep-onset stage.

In the verification, 12 persons including 6 female persons and 6 male persons were used as subjects. The ages of the subjects were 25.3±2.6 years. The body mass index (BMI), the score of morningness-eveningness questionnaire (MEQ), and the score of Pittsburgh sleep quality index (PSQI) of the 12 subjects are shown below.
BMI: 21.3±1.8.
MEQ score: 53.1±4.5.
PSQI score: 3.3±1.5.

In the case of an adult, when the BMI is 18.5 or more and less than 25, the adult is considered to have normal weight. The MEQ score is calculated within a range of 16 to 86 points, and a lower score indicates a morning type, and a higher score indicates an evening type. When the MEQ score is 42 points or more and 58 points or less, the subject is considered to be an intermediate type. The PSQI score is calculated within a range of 0 to 21 points, and when the score is 6 points or more, the subject is determined to have disturbance in sleep.

It can be seen that the 12 subjects were of normal weight and had no disturbance in sleep. 10 subjects out of the 12 subjects were intermediate type and 2 subjects were morning type. Thus, the 12 subjects can be said to be healthy subjects. In the extraction of the subjects, a questionnaire regarding the discomfort caused by the left-right difference of the binaural beats and a test regarding the ability to discriminate the left-right difference of the binaural beats were performed. As a result, the 12 subjects were determined to have no discomfort in the binaural beats and to be able to discriminate the left-right difference of the binaural beats.

Headphones were worn on the 12 subjects, and the subjects were made to nap for 90 minutes under the following four conditions.
Condition 1: Silence state.
Condition 2: The left and right ears are caused to listen to a pure sound of 250 Hz. That is, the frequencies of the binaural beats are 0 Hz.
Condition 3: The right ear is caused to listen to a pure sound of 250.25 Hz and the left ear is caused to listen to a pure sound of 250 Hz. That is, the frequencies of the binaural beats are 0.25 Hz.
Condition 4: The right ear is caused to listen to a pure sound of 251 Hz and the left ear is caused to listen to a pure sound of 250 Hz. That is, the frequencies of the binaural beats are 1 Hz.

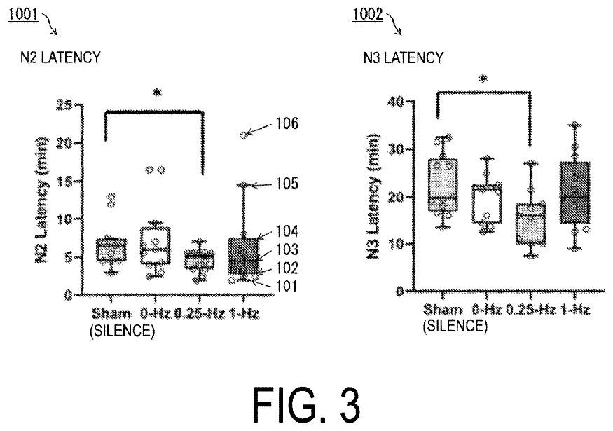

FIG. 3 is a graph showing a verification result, and reference numeral 1001 denotes data of sleep-onset latencies obtained from the 12 subjects under each of Conditions 1 to 4. The vertical axis represents the sleep-onset latency, i.e., the time (unit: minute) to reach stage 2. Reference numeral 1002 is data of deep sleep latency obtained from the 12 subjects under each of Conditions 1 to 4. The vertical axis represents the deep sleep latency, i.e., the time (unit: minute) to reach stage 3. These times were determined on the basis of brain wave data detected by an electroencephalograph worn on each of the subjects. The circles in each condition are data indicating the sleep-onset latency and the deep sleep latency for the 12 subjects.

As shown in FIG. 3, the graph of each condition is represented using a box-and-whisker plot. Each of the box-and-whisker plots is a graph obtained by extracting a minimum value 101, a first quartile 102, a median value 103, a third quartile 104, and a maximum value 105 from the data of the sleep-onset latency or deep sleep latency obtained from each subject for each condition. In the present example, data outside the range of the first quartile−the interquartile range×1.5 and data outside the range of the third quartile+the interquartile range×1.5 were specified as outliers 106. The interquartile range is a difference between the third quartile 104 and the first quartile 102.

By using each of the box-and-whisker plots, various values such as the maximum value, the minimum value, and the median value described above and distribution of data can be visually recognized. These indexes can be compared visually in the two box-and-whisker plots. Therefore, whether a difference is found between the sleep-onset latency and the deep sleep latency under the two conditions can be intuitively determined.

As shown in FIG. 3, in both the sleep-onset latency and the deep sleep latency, it can be visually recognized that the sleep time is shorter in the state in which subjects were caused to listen to the binaural beats of 0.25 Hz in Condition 3 than in the silence state in Condition 1.

Statistical verification was performed as to whether there was a difference between the sleep-onset latency and the deep sleep latency between Condition 1 and Condition 2, between Condition 1 and Condition 3, between Condition 1 and Condition 4, between Condition 2 and Condition 3, between Condition 2 and Condition 4, and between Condition 3 and Condition 4. Specifically, a significant difference test was performed for each combination of two conditions. The significant difference test is a statistical hypothesis test in which a null hypothesis is established and verified. In the present example, a Wilcoxon signed rank test, which is an example of a non-parametric test being a significant difference test between two corresponding data groups, was used as the significant difference test. However, as the significant difference test, for example, another test method such as a non-parametric test may also be used.

In this example, a null hypothesis that there was no significant difference in sleep variables between the data groups for the two conditions was established, and the significance level was set to 5%. In FIG. 3, an asterisk (*) is shown between two conditions for which the test statistic obtained as a result of performing the Wilcoxon signed rank test was less than the limit value. As shown in FIG. 3, as a result of performing the Wilcoxon signed rank test, it was recognized that there is a statistically significant difference between the sleep-onset latency and the deep sleep latency between the silence state of Condition 1 and the state in which the subject was caused to listen to the binaural beats of 0.25 Hz of Condition 3. Referring also to the box-and-whisker plot shown in FIG. 3, it was found that the sleep-onset latency and the deep sleep latency were statistically significantly reduced in the state in which subjects were caused to listen to the binaural beats of 0.25 Hz under Condition 3, as compared with the silence state under Condition 1.

As described above, it was demonstrated that the sleep-onset latency and the deep sleep latency can be shortened by causing subjects to listen to binaural beats having frequencies equal to or higher than 0.2 Hz and equal to or lower than 0.3 Hz, particularly 0.25 Hz in the sleep-onset stage.

### Example of Software Implementation

Functions of the control device 4 (hereinafter referred to as “apparatus”) can be implemented by a program for causing a computer to function as the device and for causing the computer to function as each control block (particularly, the determiner 41 and the controller 42) of the device.

In this case, the apparatus includes a computer including at least one control device (e.g., processor) and at least one storage device (e.g., memory) as hardware for executing the program. By executing the program by the control device and the storage device, the functions described in the embodiments are implemented.

The program may be recorded on one or more computer-readable non-transitory recording media. The recording media may be or may not be included in the apparatus. In the latter case, the program may be supplied to the apparatus via any wired or wireless transmission medium.

Some or all of the functions of the control blocks can be implemented by logic circuits. For example, an integrated circuit in which logic circuits functioning as the control blocks are formed is also included in the scope of the present disclosure. In addition to this, for example, a quantum computer can implement the functions of the control blocks.

The several types of processing described in the embodiments may be executed by artificial intelligence (AI). That is, each processing other than the processing of the determiner 41 may also be executed by the AI. In this case, the AI may operate in the control device such as the processor described above, or may operate in another device (e.g., an edge computer or a cloud server).

### Supplementary Note

In the present disclosure, the invention has been described above based on the drawings and examples. However, the invention according to the present disclosure is not limited to each embodiment described above. That is, the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

1 Sleep induction device
3 Beat generator
4 Control device
41 Determiner
42 Controller

1. A control device comprising:
a determiner configured to determine a stage of sleep of a subject based on biological information on the subject; and
a controller configured to control a sleep induction device comprising a beat generator configured to generate a binaural beat,
wherein the controller is configured to cause the beat generator to generate a binaural beat having a frequency lower than 1.0 Hz at least in the stage of sleep of the subject.

2. The control device according to claim 1, wherein the controller is configured to cause the beat generator to generate a binaural beat having a frequency equal to or higher than 0.2 Hz and equal to or lower than 0.3 Hz.

3. The control device according to claim 1, wherein the controller is configured to change the frequency of the binaural beat in accordance with the stage of sleep of the subject.

4. The control device according to claim 3, wherein the controller is configured to bring the frequency of the binaural beat close to a specific frequency in accordance with the stage of sleep of the subject.

5. The control device according to claim 3, wherein the controller is configured to stop the binaural beat in accordance with the stage of sleep of the subject.

6. The control device according to claim 1, wherein in response to the determiner determining that the stage of sleep of the subject is a sleep-onset stage, the controller causes the beat generator to generate the binaural beat.

7. The control device according to claim 1, wherein the controller is configured to cause the beat generator to generate the binaural beat before the subject falls asleep.

8. The control device according to claim 1, wherein when stages of sleep comprising non-REM sleep are classified into stages 1, 2, and 3 in order from the lightest stage of sleep and the determiner determines that the stage of sleep has transitioned to stage 3, the controller changes the frequency of the binaural beat or reduces a volume of a sound generated by the beat generator.

9. The control device according to claim 8, wherein in response to the determiner determining that the stage of sleep has transitioned to stage 3, the controller causes the beat generator to stop outputting the sound.

10. The control device according to claim 1, wherein the controller is configured to cause the beat generator to generate the binaural beat at a predetermined time.

11. The control device according to claim 1, wherein the controller is configured to cause the beat generator to generate the binaural beat in accordance with the biological information.

12. A sleep induction device comprising:
a beat generator configured to generate a binaural beat having a frequency lower than 1.0 Hz at least in a stage of sleep.

13. The sleep induction device according to claim 12, wherein the beat generator is configured to generate a binaural beat having a frequency equal to or higher than 0.2 Hz and equal to or lower than 0.3 Hz.
